(19) United States
(12) Patent Application Publication
(10) Pub. No.: US 2020/0390754 A1
(43) Pub. Date: Dec. 17, 2020

(54) METHODS OF ADMINISTERING TOLPERISONE

(71) Applicant: Neurana Pharmaceuticals, Inc., San Diego, CA (US)

(72) Inventors: Judy Caron, San Diego, CA (US); Jeff Raser, San Diego, CA (US)

(21) Appl. No.: 16/764,609

(22) PCT Filed: Nov. 19, 2018
(86) PCT No.: PCT/US2018/061799

§ 371 Date: May 15, 2020

Related U.S. Application Data
(60) Provisional application No. 62/587,950, filed on Nov. 17, 2017.

(51) Int. Cl. A61K 31/4453; A61K 31/135; A61P 25/04; A61P 21/02; A61K 9/00

(52) U.S. Cl. CPC A61K 31/4453; A61K 31/135; A61K 9/0053; A61P 21/02; A61P 25/04

(57) ABSTRACT

The present disclosure provides methods of treating a subject with a daily regimen of tolperisone effective against muscle spasm or spastic syndrome. The regimen is effective without impairing cognitive function, and may improve cognitive function, as measured by various cognitive and alertness tests.

### CROSS REFERENCE TO RELATED APPLICATIONS

This application is a U.S. national stage application of International Application No. PCT/US2018/061799, filed Nov. 19, 2018, which claims the benefit of priority to U.S. Provisional Application No. 62/587,950, filed on Nov. 17, 2017, the contents of which are hereby incorporated by reference in their entirety.

### FIELD OF THE INVENTION

The present disclosure relates to methods of administering tolperisone. The disclosure further relates to methods of treating a subject with a daily regimen of tolperisone effective against muscle spasm or spastic syndrome without impairing the subject's cognitive function. The regimen may also be effective to treat muscle spasm or spastic syndrome with concomitant improvement in the subject's cognitive function.

### BACKGROUND

Tolperisone is a centrally-acting muscle relaxant that has been used for the symptomatic treatment of spasticity and muscle spasm (Martindale, The Extra Pharmacopoeia, 30th ed., p. 1211). Tolperisone has also been used in the treatment of conditions, which include dysmenorrhea, climacteric complaints, lockjaw, and neurolatyrism.

The chemical structure of tolperisone is shown below.

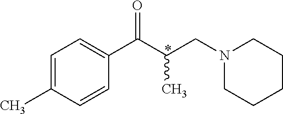 [SMILES: Cc1ccc(C(=O)C(C)CN2CCCCC2)cc1]

As can be seen by the foregoing structure, tolperisone contains a chiral center (as indicated by the asterisk). The chiral separation of tolperisone into its R(−) and S(+) enantiomers has been described (See, for example, JP-A-53-40779).

Racemic tolperisone is commercially available as the hydrochloride salt and is sold under trade names such as Mydeton®, Mydocalm®, Midocalm® and Muscalm®.

Tolperisone has been shown to exhibit membrane-stabilizing effects in the central and peripheral nervous system (Ono, H., et al, J. Pharmacobio. Dynam. 1984, 7, 171-178). Tolperisone hydrochloride is used for improving not only different symptoms related to spastic paralysis, but also for improving muscle tone, which originates from diseases or conditions such as cervical syndrome, inflammation of the joints, and back pain. The use of tolperisone for treating neuropathic pain and pain associated with various nervous system disorders has also been described (see, for example, U.S. Patent Application No. 2006/0004050).

Generally, centrally acting muscle relaxants reduce the increased muscle tonus and are typically sedative when used. For example, physicians cite drowsiness as their most common concern when prescribing skeletal muscle relaxants, like cyclobenzaprine hydrochloride (sold under the names FLEXERIL® and AMRIX®).

Thus, there is a need for treatment methods that allow a physician to determine a daily treatment regimen of tolperisone that is most effective for treating muscle spasm or spastic syndrome without impairing the patient's cognitive function. In addition to being a highly effective, non-sedating, drug for the treatment of muscle spasm or spastic syndrome, the present inventors have now surprisingly found that tolperisone improves cognitive function in tests measuring the alertness of the subject following administration of the drug. One useful measure of alertness described herein is the effect of a drug on the ability of the subject to stay alert during a driving test.

### SUMMARY OF THE INVENTION

In one aspect, the present disclosure provides a method of treating a patient with muscle spasm or spastic syndrome, wherein based on the patient's routine daily activities, an optimal daily treatment regimen of tolperisone or a pharmaceutically acceptable salt thereof is selected to treat muscle spasm or spastic syndrome without impairing cognitive function.

In another aspect, the present disclosure provides a method of treating a patient with muscle spasm or spastic syndrome, comprising administering to the patient a therapeutically effective amount of tolperisone or a pharmaceutically acceptable salt thereof, and wherein an improvement is also seen in the patient's cognitive function.

In a further aspect, the present disclosure provides a method of treating a patient with muscle spasm or spastic syndrome comprising (1) assessing the patient's routine daily activities, (2) determining if one or more of said activities could be, or is/are being, compromised by administering a drug effective to treat muscle spasm or spastic syndrome, but also known to have a sedative effect and (3) substituting said drug with tolperisone or a pharmaceutically acceptable salt thereof, and administering tolperisone or a pharmaceutically acceptable salt thereof in an amount effective to treat muscle spasm or spastic syndrome without compromising the patient's routine daily activities.

In certain embodiments, the assessment of a patient results in the physician discontinuing treatment with a muscle relaxant that has a sedative effect (e.g. cyclobenzaprine) and instead prescribing tolperisone to treat muscle spasm or spastic syndrome.

In certain embodiments, the muscle spasm or spastic syndrome is post-stroke spasticity.

In certain embodiments, the muscle spasm or spastic syndrome is one or more acute musculoskeletal conditions.

In certain further embodiments, muscle spasm or spastic syndrome is acute muscle spasm of the neck and/or back.

In certain further embodiments, tolperisone provides relief of neck and/or back pain due to muscle spasm of acute onset.

### DETAILED DESCRIPTION OF THE DRAWINGS

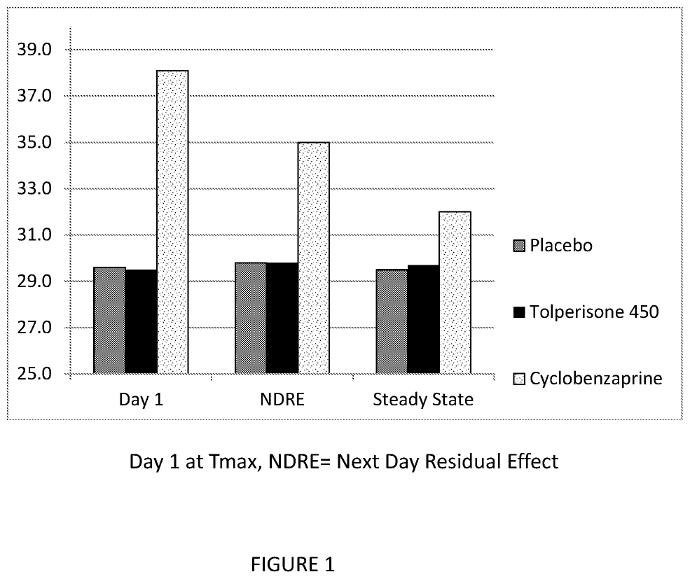

FIG. 1 describes the results of a driving simulation where the primary endpoint is the Standard Deviation of Lateral Position (SDLP). In this test, tolperisone and placebo gave equivalent results confirming tolperisone was non-sedating. However, cyclobenzaprine administration induced a significantly impaired effect.

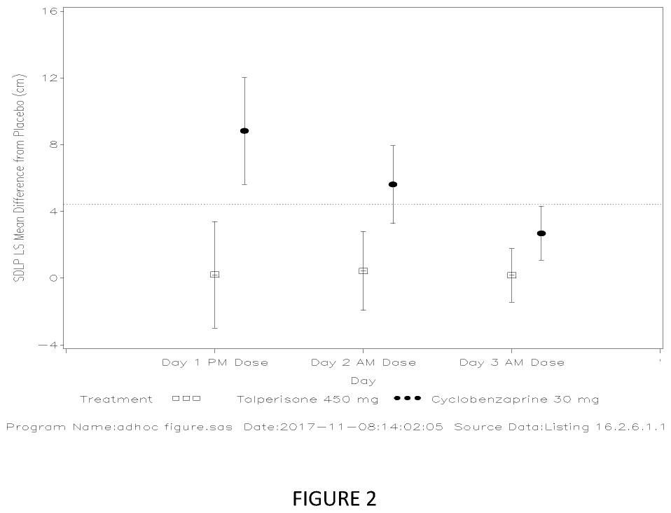

FIG. 2 depicts further data from the driving simulation in which the mean difference in effect from placebo is provided for tolperisone and cyclobenzaprine.

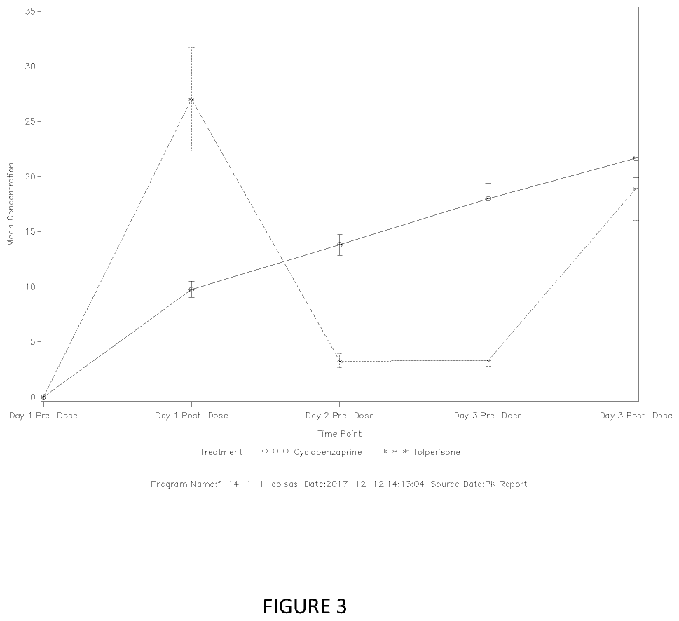

FIG. 3 depicts the mean plasma concentration of tolperisone and cyclobenzaprine following administration over a three-day period.

### DETAILED DESCRIPTION

All publications, patents and patent applications cited herein, whether supra or infra, are hereby incorporated by reference in their entirety for all purposes.

### Definitions

It must be noted that, as used in this specification, the singular forms “a,” “an,” and “the” include plural referents unless the context clearly dictates otherwise.

In describing and claiming the present invention, the following terminology will be used in accordance with the definitions described below.

The term “tolperisone”, as well as reference to other chemical compounds herein, includes the compound in any of its pharmaceutically acceptable forms, including isomers such as diastereomers and enantiomers, salts, solvates, and polymorphs, particular crystalline forms, as well as racemic mixtures and pure isomers of the compounds described herein, where applicable.

“Pharmaceutically acceptable excipient or carrier” refers to an excipient that may optionally be included in the compositions of the invention and that causes no significant adverse toxicological effects to the patient upon administration.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

“Optional” or “optionally” means that the subsequently described circumstance may or may not occur, so that the description includes instances where the circumstance occurs and instances where it does not.

“Substantially absent” or “substantially free” of a certain feature or entity means nearly totally or completely absent the feature or entity. As used herein, a tolperisone formulation that is substantially absent or substantially free of 4-MMPPO contains less than about 10 ppm 4-MMPPO.

A tolperisone composition that has been stored under “dry conditions” is one that has been stored under controlled humidity conditions (5-25 percent relative humidity) and at temperatures ranging from about 18-25° C. The tolperisone composition may be the active pharmaceutical ingredient (API), or a pharmaceutical composition (powder or the like) comprising tolperisone and one or more pharmaceutically acceptable excipients, or a finished product, for example, a capsule, tablet, etc. The composition is contained in a sealed container such as a bottle, blister, pouch, or a combination thereof. The composition may also be stored in the presence of a dessicant, such as silica, typically encased in a pack suitable for absorption of water vapor.

“Anhydrous” refers to a material that is substantially absent water.

The terms “subject”, “individual” or “patient” are used interchangeably herein and refer to a vertebrate, preferably a mammal. Mammals include, but are not limited to, humans.

Additional definitions can be found in the sections that follow.

### Tolperisone

Tolperisone for use as part of the present disclosure may be obtained commercially, or can be synthesized by a variety of methods known in the art. See, e.g., U.S. Patent Application Publication No. 2006/0041141; Ditriech et al. (1999) J. Labeled Cpd. Radiopharm, 42:1125-1134; Jap. Pat. No. 04005283 19920109; Jap. Pat. No. 54032480 19790309; Jap. Pat. No. 54036274 19790316; Jap. Pat. No. 54030178 19790306; Jap. Pat. No. 54027571 19790301; Kazuharu et al. (1994) Chem. Pharm. Bulletin 42(8) 1676; Jap. Pat. No. 20,390 (1965); and Hung. Pat. No. 144,997 (1956), each incorporated herein by reference in its entirety.

Commercial formulations of tolperisone, such as Mydeton® and Mydocalm®, and tolperisone prepared according to most known synthetic methods, possess levels of 2-methyl-1-(4-methylphenyl)propenone (4-MMPPO) in excess of 100 ppm. Due to the genotoxic side effects associated with 4-MMPPO, tolperisone is preferably prepared and formulated for use herein in accordance with other methods referenced and described hereinafter. Such methods produce tolperisone substantially free of 4-MMPPO.

U.S. Pat. No. 9,675,598 (which is hereby incorporated by reference in its entirety for all purposes) discloses methods of detecting levels of 4-MMPPO below about 0.001% weight (10 ppm). It is believed that the method is capable of detecting levels of 4-MMPPO down to at least 0.5 ppm.

U.S. Pat. No. 9,675,598 discloses methods of preparing tolperisone formulations that are substantially free of 4-MMPPO. Thus, tolperisone formulations that contain less than about 10 ppm 4-MMPPO may be prepared, for example, by recrystallization and acid treatment (see Examples 1-5 hereinafter).

Preferably, tolperisone and tolperisone compositions as described herein are stored under dry conditions. Dry conditions as used herein refers to a temperature ranging from about 18 to 23° C. and a relative humidity of 5-25%. The compositions may also be stored in the presence of a desiccant, such as silica, typically encased in a pack suitable for absorption of water vapor.

In a preferred embodiment, a tolperisone composition herein comprises tolperisone in the form of an acid addition salt (e.g. racemic tolperisone hydrochloride). In another preferred embodiment, a tolperisone composition herein will also comprise an additional amount of an acidic additive or excipient to establish an environment that is more acidic than that provided by tolperisone in the form of an acid addition salt. Such additives include acetic acid, succinic acid, adipic acid, propionic acid, citric acid, toluenesulfonic acid, methanesulfonic acid, and the like. Preferred acids are di-acids or greater (e.g., di-acids, tri-acids, etc.), having more than one acidic proton. Preferably, acids for use as stabilizers for compositions of tolperisone will possess a pKa of less than about 3. Preferably, the acid is anhydrous. Particularly preferred acids include citric acid and succinic acid.

Tolperisone may also be combined within a glassy matrix; glass formers are well known in the art, and may be effective in preventing chemical degradation of tolperisone, particularly degradation that results in the formation of 4-MMPPO.

### Tolperisone Uses and Treatment Regimens

Tolperisone is a centrally-acting muscle relaxant that acts on the central nervous system and is used mainly for the treatment of elevated muscle tone and tension, as well as for certain circulatory problems in the extremities. Tolperisone has been found to reduce experimental hypertonia and decerabration rigidity, as well as inhibit reticulospinal reflex facilitation without affecting cortical functions. It also improves peripheral blood flow (Toperin® Package Insert).

Tolperisone is useful in treating a number of conditions. For example, tolperisone may be administered to a subject suffering from one of more of the following conditions including: muscle spasm, spastic syndromes, muscle soreness, myotonia, dysmenorrhea, climacteric complaints, lockjaw, neurolatyrism, osteoarthritis or rheumatoid arthritis (when administered in combination with a non-steroidal anti-inflammatory drug), rheumatic diseases, fibromyalgia syndrome, occupational and sport-related stress, back pain, spasticity caused by neurological diseases, multiple sclerosis, myelopathy, encephalomyelitis, stroke, muscular hypertension, muscular contracture, spinal automatism, obliterative vascular diseases (e.g., obliterative arteriosclerosis, diabetic angiopathy, obliterative thromboangitis, Raynaud's disease, diffuse scleroderma), disorders due to injured innervation of the vessels (acrocyanosis, intermittent angioneurotic dysbasis), neuropathic pain, and in individual cases, post-thrombotic venous and lymphatic circulation disorders, diabetic neuropathy, post-herpetic neuralgia, and crural ulcer (Myolax® Package insert).

Subjects to whom tolperisone may be administered include both children (aged three months to 18 years), and adults (18 years and older).

When treating muscle spasm or spastic syndrome, the inventors have now found that the optimal daily regimen of tolperisone can be accurately calculated based on an understanding of the subject's routine daily activities, and measuring parameters related to the subject's cognitive function. For example, cognitive function may be assessed using a functional test, such as the Digit Symbol Substitution test (DSST). Examples of routine daily activities measured include driving ability, child care competence and routine daily functioning.

The subject's level of alertness may conveniently be measured by assessing their driving performance. In one aspect, the alertness of the subject may be assessed by measuring driving parameters prior to and subsequent to the administration of tolperisone. Driving performance may be measured using over-the-road driving tests, or more conveniently using a computer simulation, such as, for example, using a driving scenario (e.g. Country Vigilance-Divided Attention driving scenario; CVDA) on a CRDDS-MiniSim. Another measure of alertness (related to a subjective level of sleepiness) is the Karolinska Sleepiness Scale (KSS), which provides an assessment of alertness/sleepiness at a particular point in time. The KSS has been found to correlate with electroencephalograms and behavioral variables.

The inventors have also surprisingly found that tolperisone administration has a positive effect on cognitive function, and in particular improves the subject's alertness when tested, such as when driving performance is tested and reaction time is measured. For example, according to Table 8 in Example 8, there was no statistically significant difference in the majority of secondary driving endpoint measures when tolperisone is compared to placebo in a computer-simulated driving test. This confirms that, unlike other muscle relaxants such as cyclobenzaprine, tolperisone has no significant sedative effect on the subjects and is non-drowsy. However, in one parameter tested, namely reaction time, tolperisone demonstrated a statistically significant improvement compared to treatment with placebo or cyclobenzaprine. Reaction time is a particularly useful measure in this test of the alertness of the subject. One might expect that the reaction time following administration of tolperisone would be superior to the reaction time following administration of cyclobenzaprine. However, it was totally unexpected that tolperisone demonstrated a superior reaction time result versus treatment with placebo.

Thus, in some embodiments, the present disclosure provides a method of treating a patient with muscle spasm or spastic syndrome, wherein based on the patient's routine daily activities, an optimal daily treatment regimen of tolperisone or a pharmaceutically acceptable salt thereof is selected to treat muscle spasm or spastic syndrome without impairing cognitive function.

In some embodiments, the optimal daily treatment regimen of tolperisone or a pharmaceutically acceptable salt thereof is selected to treat muscle spasm or spastic syndrome without impairing cognitive function as characterized by no increase in Karolinska Sleepiness Scale score compared to prior to treatment. In some embodiments, the optimal daily treatment regimen of tolperisone or a pharmaceutically acceptable salt thereof is selected to treat muscle spasm or spastic syndrome without impairing cognitive function as characterized by an increase of not more than 1 point in Karolinska Sleepiness Scale score compared to prior to treatment. In some embodiments, the optimal daily treatment regimen of tolperisone or a pharmaceutically acceptable salt thereof is selected to treat muscle spasm or spastic syndrome without impairing cognitive function as characterized by an increase of not more than 2 points in Karolinska Sleepiness Scale score compared to prior to treatment. In some embodiments, the optimal daily treatment regimen of tolperisone or a pharmaceutically acceptable salt thereof is selected to treat muscle spasm or spastic syndrome without impairing cognitive function as characterized by an increase of not more than 3 points in Karolinska Sleepiness Scale score compared to prior to treatment.

In some embodiments, the optimal daily treatment regimen of tolperisone or a pharmaceutically acceptable salt thereof is selected to treat muscle spasm or spastic syndrome without impairing cognitive function as characterized by less than about a 10% increase in Standard Deviation of Lateral Position (SDLP) in a computer simulation compared to prior to treatment. In some embodiments, the optimal daily treatment regimen of tolperisone or a pharmaceutically acceptable salt thereof is selected to treat muscle spasm or spastic syndrome without impairing cognitive function as characterized by less than about a 5% increase in SDLP in a computer simulation compared to prior to treatment. In some embodiments, the optimal daily treatment regimen of tolperisone or a pharmaceutically acceptable salt thereof is selected to treat muscle spasm or spastic syndrome without impairing cognitive function as characterized by no significant increase in SDLP in a computer simulation compared to prior to treatment. In some embodiments, the optimal daily treatment regimen of tolperisone or a pharmaceutically acceptable salt thereof is selected to treat muscle spasm or spastic syndrome without impairing cognitive function as characterized by no significant increase in SDLP in a computer simulation compared to a placebo treated patient.

In some embodiments, the optimal daily treatment regimen of tolperisone or a pharmaceutically acceptable salt thereof is selected to treat muscle spasm or spastic syndrome without impairing cognitive function as characterized by less than about a 10% increase in lane exceedance in a computer simulation compared to prior to treatment. In some embodiments, the optimal daily treatment regimen of tolperisone or a pharmaceutically acceptable salt thereof is selected to treat muscle spasm or spastic syndrome without impairing cognitive function as characterized by less than about a 5% increase in lane exceedance in a computer simulation compared to prior to treatment. In some embodiments, the optimal daily treatment regimen of tolperisone or a pharmaceutically acceptable salt thereof is selected to treat muscle spasm or spastic syndrome without impairing cognitive function as characterized by no significant increase in lane exceedance in a computer simulation compared to prior to treatment. In some embodiments, the optimal daily treatment regimen of tolperisone or a pharmaceutically acceptable salt thereof is selected to treat muscle spasm or spastic syndrome without impairing cognitive function as characterized by no significant increase in lane exceedance in a computer simulation compared to a placebo treated patient.

In some embodiments, the optimal daily treatment regimen of tolperisone or a pharmaceutically acceptable salt thereof is selected to treat muscle spasm or spastic syndrome without impairing cognitive function as characterized by less than about a 10% increase in total collisions in a computer simulation compared to prior to treatment. In some embodiments, the optimal daily treatment regimen of tolperisone or a pharmaceutically acceptable salt thereof is selected to treat muscle spasm or spastic syndrome without impairing cognitive function as characterized by less than about a 5% increase in total collisions in a computer simulation compared to prior to treatment. In some embodiments, the optimal daily treatment regimen of tolperisone or a pharmaceutically acceptable salt thereof is selected to treat muscle spasm or spastic syndrome without impairing cognitive function as characterized by no significant increase in total collisions in a computer simulation compared to prior to treatment. In some embodiments, the optimal daily treatment regimen of tolperisone or a pharmaceutically acceptable salt thereof is selected to treat muscle spasm or spastic syndrome without impairing cognitive function as characterized by no significant increase in total collisions in a computer simulation compared to a placebo treated patient.

In other embodiments, the present disclosure provides a method of treating a patient with muscle spasm or spastic syndrome, comprising administering to the patient a therapeutically effective amount of tolperisone or a pharmaceutically acceptable salt thereof, and wherein an improvement is also seen in the patient's cognitive function (e.g. an improvement in alertness).

In some embodiments, the improvement in the patient's cognitive function is characterized by an improvement of at least about 20% in reaction time (i.e. the time to respond to a stimulus) compared to reaction time measured prior to treatment with tolperisone or a pharmaceutically acceptable salt thereof. In some embodiments, the improvement in the patient's cognitive function is characterized by an improvement of at least about 10% in reaction time compared to reaction time measured prior to treatment with tolperisone or a pharmaceutically acceptable salt thereof. In some embodiments, the improvement in the patient's cognitive function is characterized by an improvement of at least about 5% in reaction time compared to reaction time measured prior to treatment with tolperisone or a pharmaceutically acceptable salt thereof. In some embodiments, the improvement in the patient's cognitive function is characterized by a significant improvement in reaction time when a patient treated with tolperisone or a pharmaceutically acceptable salt thereof compared to the same patient treated with a placebo.

In some embodiments, reaction time is measured using a car-driving stimulus, e.g. measuring time to start to change car direction to avoid a collision, time to start to brake to reduce speed and/or time to start to move back into the correct lane following lane exceedance. The measurements may conveniently be taken during a computer-simulated driving test.

Furthermore, the inventors have found that the administration of tolperisone at therapeutically effective doses does not produce a hangover effect the morning following nighttime dosing. This offers substantial advantages in the treatment of subjects with muscle spasm or spastic syndrome over established treatment regimens with muscle relaxants that are sedating (for example, when cyclobenzaprine is administered to the subject).

In some embodiments, the patient experiences no substantial sedation following treatment with tolperisone compared to prior to treatment at steady state blood plasma concentrations. In other embodiments, the patient experiences no substantial sedation following treatment with tolperisone compared to prior to treatment at T max blood plasma concentrations.

In some embodiments, the patient experiences no substantial sedation following treatment with tolperisone that is characterized by no increase in Karolinska Sleepiness Scale score compared to prior to treatment. In some embodiments, the patient experiences no substantial sedation following treatment with tolperisone that is characterized by an increase of not more than 1 point in Karolinska Sleepiness Scale score compared to prior to treatment. In some embodiments, the patient experiences no substantial sedation following treatment with tolperisone that is characterized by an increase of not more than 2 points in Karolinska Sleepiness Scale score compared to prior to treatment. In some embodiments, the patient experiences no substantial sedation following treatment with tolperisone that is characterized by an increase of not more than 3 points in Karolinska Sleepiness Scale score compared to prior to treatment.

In some embodiments, the present disclosure provides a method of treating a patient with muscle spasm or spastic syndrome comprising (1) assessing the patient's routine daily activities, (2) determining if one or more of said activities could be, or is/are being, compromised by administering a drug effective to treat muscle spasm or spastic syndrome, but also known to have a sedative effect (e.g. cyclobenzaprine) and (3) substituting said drug with tolperisone or a pharmaceutically acceptable salt thereof, and administering tolperisone or a pharmaceutically acceptable salt thereof in an amount effective to treat muscle spasm or spastic syndrome without compromising the patient's routine daily activities.

In other embodiments, the present disclosure provides a method of treating a patient with muscle spasm or spastic syndrome, comprising administering to the patient a therapeutically effective amount of tolperisone or a pharmaceutically acceptable salt thereof, and wherein the patient was previously treated with a muscle relaxant (e.g. cyclobenzaprine) but treatment was discontinued because of the muscle relaxant's sedative effect.

Examples of a patient's routine daily activities may preferably include driving. In a particular embodiment of the present disclosure, the effect of a muscle relaxant on driving performance is determined using a computer-simulated driving test.

In one embodiment, the present disclosure provides a method of treating one or more of the muscle conditions described herein comprising administering a therapeutically effective amount of tolperisone or a pharmaceutically acceptable salt or hydrate thereof, to a patient in need thereof, wherein after said treating the patient experiences no substantial change in driving performance compared to prior to said treating.

Driving performance may be measured using any suitable method known to those skilled in the art. In certain embodiments, driving performance is measured by standard deviation of lateral position in a computer simulation, lane exceedance in a computer simulation or total collisions in a computer simulation.

In some embodiments, the patient experiences no substantial change in driving performance before and after treatment, at steady state blood plasma concentrations. In still other embodiments, the patient experiences no substantial change in driving performance before and after treatment at T max blood plasma concentrations.

In some embodiments, patient experiences no substantial change in driving performance before and after treatment that is characterized by less than about a 10% increase in Standard Deviation of Lateral Position (SDLP) in a computer simulation compared to prior to said treating. In some embodiments, patient experiences no substantial change in driving performance before and after treatment that is characterized by less than about a 5% increase in SDLP in a computer simulation compared to prior to said treating. In some embodiments, patient experiences no substantial change in driving performance before and after treatment that is characterized by no significant increase in SDLP in a computer simulation compared to prior to said treating. In some embodiments, patient experiences no substantial change in driving performance before and after treatment that is characterized by no significant increase in SDLP in a computer simulation compared to a placebo treated patient.

In some embodiments, patient experiences no substantial change in driving performance before and after treatment that is characterized by less than about a 10% increase in lane exceedance in a computer simulation compared to prior to said treating. In some embodiments, patient experiences no substantial change in driving performance before and after treatment that is characterized by less than about a 5% increase in lane exceedance in a computer simulation compared to prior to said treating. In some embodiments, patient experiences no substantial change in driving performance before and after treatment that is characterized by no significant increase in lane exceedance in a computer simulation compared to prior to said treating. In some embodiments, patient experiences no substantial change in driving performance before and after treatment that is characterized by no significant increase in lane exceedance in a computer simulation compared to a placebo treated patient.

In some embodiments, patient experiences no substantial change in driving performance before and after treatment that is characterized by less than about a 10% increase in total collisions in a computer simulation compared to prior to said treating. In some embodiments, patient experiences no substantial change in driving performance before and after treatment that is characterized by less than about a 5% increase in total collisions in a computer simulation compared to prior to said treating. In some embodiments, patient experiences no substantial change in driving performance before and after treatment that is characterized by no significant increase in total collisions in a computer simulation compared to prior to said treating. In some embodiments, patient experiences no substantial change in driving performance before and after treatment that is characterized by no significant increase in total collisions in a computer simulation compared to a placebo treated patient.

In some embodiments, the treated patient experiences no next day residual effect (i.e., no hangover effect).

In some embodiments, the one or more of the muscle conditions described herein is a muscle spasm or spastic syndrome. In certain further embodiments, the muscle spasm or spastic syndrome is post-stroke spasticity.

In other embodiments, the muscle spasm or spastic syndrome is one or more acute musculoskeletal conditions. In certain further embodiments, the one or more acute musculoskeletal conditions is acute muscle spasm of the neck and/or back.

In further embodiments, tolperisone provides relief of neck and/or back pain due to muscle spasm of acute onset.

In some embodiments, tolperisone is co-administered with an analgesic to treat neck and/or back pain due to muscle spasm. Suitable analgesics will be well known in the art and include anti-inflammatory agents such as NSAIDs (e.g. aspirin, celecoxib, diclofenac, ibuprofen, indomethacin, tolmetin and naproxen) and opioids (e.g. oxycodone or hydrocodone). Tolperisone and the analgesic may be administered in a single dosage form or, preferably, in separate dosage forms, simultaneously or separately over any conveniently time spacing between doses.

In some embodiments, tolperisone may exhibit an additive or synergistic effect with the analgesic. This may be particularly advantageous when the analgesic is an opioid and may lead to an opioid-sparing effect. Thus, a particular embodiment of the present disclosure provides a method of treating neck and/or back pain due to muscle spasm by administering to the subject a therapeutically effective amount of tolperisone in combination therapy with a pain medication (e.g. an opioid drug), where the pain medication (e.g. an opioid drug) is administered at a reduced dose compared to the dose required to effectively treat a patient with painful muscle spasm of the back or neck who is not also administered tolperisone.

The driving study described herein (e.g. a computer-simulated driving study) is a particularly useful test to enable regulatory authorities to determine if a drug in development is suitable for progression towards approval for marketing, based on a combination of its effectiveness to treat muscle spasm or spastic syndrome and its sedative effect as measured by the driving study. The study is a very effective model when used with an active comparator, which has known sedative effects.

Thus, one embodiment provides a method of determining the suitability of a drug for treating a patient suffering from muscle spasm or spastic syndrome, comprising conducting a driving simulation study and comparing the sedative effect of the drug against a standard therapy for treating muscle spasm or the spastic syndrome (e.g. cyclobenzaprine).

### Tolperisone Doses

Generally, a therapeutically effective amount of tolperisone for an adult will range from a total daily dosage of between about 10 and about 3000 mg/day, preferably, in an amount between 25-2000 mg/day, more preferably, in an amount between about 50 and about 1800 mg/day. Typical dosage ranges for adults include total daily dosage ranges from about 150 and about 1200 mg/day, preferably from about 450 to about 900 mg/day to treat spasticity and from about 150 to about 450 mg/day to treat muscle spasm. Preferred in certain embodiments are divided dosages over the course of a day, e.g. a recommended daily dose divided into five doses, or four doses, or three doses, or two doses. Preferred dosage amounts include dosages from about 50 mg to 450 mg administered three times daily. Dosage amounts may conveniently be selected from 50 mg/day, 100 mg/day, 150 mg/day, 200 mg/day, 250 mg/day, 300 mg/day, 350 mg/day, 400 mg/day, 450 mg/day, 500 mg/day, 550 mg/day, 600 mg/day or more.

Depending upon the dosage amount and precise condition to be treated, administration can be over a time course of one day to several days, weeks, months, or longer. Illustrative dosing regimens will last a period of at least about a day, a week, from about 1-4 weeks, from 1-3 months, from 1-6 months, from 1-50 weeks, from 1-12 months, or longer.

In some embodiments, the tolperisone is administered for a period of about 1 week, about 2 weeks, about 3 weeks, about 4 weeks, about 6 weeks, about 8 weeks, about 10 weeks, about 12 weeks, about 24 weeks or about 50 weeks.

In some embodiments, the tolperisone is administered one time per day, two times per day, three times per day or four times per day, and particularly three times per day.

Dosage amounts for children ranging in age from 3 months to 18 years in age range from about 1-25 mg/kg/day, preferably from about 2-15 mg/day, in from about 2-4 divided doses, preferably 3 doses. Exemplary recommended dosage ranges for children include 5-10 mg/kg/day and from 2-4 mg/kg/day, in 2-3 divided doses.

In one particular embodiment of the present disclosure, tolperisone is administered three times daily (TI D) in 50 mg, 100 mg, 150 mg or 200 mg unit doses to a total daily dose of 150 to 600 mg/day.

Additionally, the dosage of a tolperisone composition administered to a subject can be limited to prevent overexposure of the subject to 4-MMPPO. Therefore, in another particular embodiment, the total daily dose results in a daily exposure to the patient of less than about 20 μg, preferably less than about 10 μg, and more preferably less than about 1.5 μg of 4-MMPPO.

### Tolperisone Formulations

In addition to comprising tolperisone, a formulation of the invention may optionally contain one or more additional components.

A composition of the invention may comprise, in addition to tolperisone, one or more pharmaceutically acceptable excipients or carriers. Exemplary excipients include, without limitation, polyethylene glycol (PEG), hydrogenated castor oil (HCO), cremophors (polyethoxylated castor oil), carbohydrates, starches (e.g., corn starch), inorganic salts, antimicrobial agents, antioxidants, binders/fillers, surfactants, lubricants (e.g., calcium or magnesium stearate), glidants such as talc, disintegrant (e.g., sodium starch glycolate, cross-linked povidone, cross-linked sodium carboxymethyl cellulose), diluents, buffers, acids, bases, film coats, combinations thereof, and the like.

A composition of the invention may include one or more carbohydrates such as a sugar, a derivatized sugar such as an alditol, aldonic acid, an esterified sugar, and/or a sugar polymer. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

Also suitable for use in the compositions of the invention are potato and corn-based starches such as sodium starch glycolate and directly compressible modified starch.

Further representative excipients include inorganic salts or buffers such as citric acid, sodium chloride, potassium chloride, sodium sulfate, potassium nitrate, sodium phosphate monobasic, sodium phosphate dibasic, and combinations thereof.

A tolperisone composition may also include an antimicrobial agent, e.g., for preventing or deterring microbial growth. Non-limiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

A composition as provided herein may also contain one or more antioxidants. Antioxidants are used to prevent oxidation, thereby preventing the deterioration of the tolperisone or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

Additional excipients include surfactants such as polysorbates, e.g., “Tween 20” and “Tween 80,” and pluronics such as F68 and F88 (both of which are available from BASF, Mount Olive, N.J.), sorbitan esters, lipids (e.g., phospholipids such as lecithin and other phosphatidylcholines, and phosphatidylethanolamines), fatty acids and fatty esters, steroids such as cholesterol, and chelating agents, such as EDTA, zinc and other such suitable cations.

Further, as described previously, a composition of the invention may optionally include one or more acids. Non-limiting examples of acids that can be used include those acids selected from the group consisting of hydrochloric acid, acetic acid, phosphoric acid, citric acid, succinic acid, adipic acid, propionic acid, toluenesulfonic acid, methanesulfonic acid, malic acid, lactic acid, formic acid, trichloroacetic acid, nitric acid, perchloric acid, phosphoric acid, sulfuric acid, fumaric acid, and combinations thereof.

In a preferred embodiment, a composition as provided herein is absent a basic component.

The amount of any individual excipient in the composition will vary depending on the role of the excipient, the dosage requirements of the active agent (i.e., tolperisone), and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5% to about 98% by weight, more preferably from about 15 to about 95% by weight of the excipient. In general, the amount of excipient present in a tolperisone composition of the invention is selected from the following: at least about 2%, 5%, 10%, 15%, 20%, 25%, 30%, 35%, 40%, 45%, 50%, 55%, 60%, 65%, 70%, 75%, 80%, 85%, 90%, or even 95% by weight.

Exemplary formulations for administration include those currently on the market, e.g., Mydeton®, Mydocalm®, Midocalm® and Muscalm®, and similar such formulations.

Tolperisone may be provided in a sustained-release formulation. See, e.g., Example 9, and International Patent Publication No. WO 2005/094825. Controlled or sustained-release formulations are typically prepared by incorporating tolperisone into a carrier or vehicle such as liposomes, nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures.

One exemplary controlled release formulation includes a mixture of anionic and cationic polymers, such as Eudragit RS, Eudragit L and Eudragit S. Additionally, tolperisone can be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; and McGee et al., J. Microencap. (1996). Tablets or caplets may also be coated with water insoluble polymers, e.g, Aquacoat® and Eudragit®.

The foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

### Tolperisone Dosage Forms

The tolperisone described herein may be formulated into any form suitable for administration. Oral dosage forms include tablets, lozenges, capsules, syrups, oral suspensions, emulsions, granules, and pellets. Alternative formulations include aerosols, transdermal patches, gels, creams, ointments, suppositories, powders or lyophilates that can be reconstituted, as well as liquids. With respect to liquid pharmaceutical compositions, solutions and suspensions are envisioned. Preferably, tolperisone is provided in a form suitable for oral administration.

For example, tablets can be made by compression or molding, optionally with one or more accessory ingredients or additives. Compressed tablets are prepared, for example, by compressing in a suitable tabletting machine, the active ingredients in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) and/or surface-active or dispersing agent.

Molded tablets are made, for example, by molding in a suitable tabletting machine, a mixture of powdered compounds moistened with an inert liquid diluent. The tablets may optionally be coated or scored, and may be formulated so as to provide slow or controlled release of the active ingredients, using, for example, hydroxypropylmethyl cellulose in varying proportions to provide the desired release profile. Tablets may optionally be provided with a coating, such as a thin film, sugar coating, or an enteric coating to provide release in parts of the gut other than the stomach. Processes, equipment, and toll manufacturers for tablet and capsule making are well-known in the art.

The compositions of the present invention may also be prepared in a form suitable for veterinary applications.

### Tolperisone Administration

Methods of administering therapeutic formulations of tolperisone include, but are not limited to, oral, parenteral (including intra-arterial, intraspinal, intramuscular, intraperitoneal, intravenous, subcutaneous, intramuscular, and intradermal), rectal, nasal, topical (including transdermal, aerosol, buccal and sublingual), vaginal, intrathecal, and inhalation routes. The preferred route will vary with the condition and age of the recipient, the particular condition treated, and if tolperisone is used in combination with an analgesic, the specific combination of drugs employed.

Preferred routes of administration are intramuscular, intravenous, and oral. In a particularly preferred embodiment, tolperisone is administered orally.

### EXAMPLES

The following examples illustrate certain aspects and advantages of the present invention, however, the present invention is in no way considered to be limited to the particular embodiments described below.

The practice of the invention will employ, unless otherwise indicated, techniques of pharmaceutical formulation, separations, pharmacology, and the like, which are within the skill of the art, based upon the guidance provided herein. See, for example, Handbook of Pharmaceutical Manufacturing Formulations, S. K. Niazi (ed.), CRC Press, 2004; Goodman & Gilman, The Pharmacological Basis of Therapeutics, 9th Edition, Hardman, J. G., Gilman, A. G., Limbird, L. E. (eds.), McGraw-Hill, New York, 1995; Basic and Clinical Pharmacology, 18th Edition, Katzung, B. G. (ed.), Appleton & Lange, Norwalk, Conn., 2001.

In the following examples, efforts have been made to ensure accuracy with respect to numbers used (e.g., amounts, temperatures, etc.) but some experimental error and deviation should be accounted for. Each of the following examples is considered to be instructive to one of ordinary skill in the art for carrying out one or more of the embodiments described herein.

### Example 1: Standard Recrystallization of Tolperisone

Tolperisone containing 4-MMPPO at levels of greater than 0.5% by weight (referred to hereinafter as ‘Crude tolperisone’) was employed as the starting material for the recrystallization experiments hereinafter. Crude tolperisone was dissolved in an 85:15 (v/v) mixture of 2-butanone (methyl ethyl ketone) and isopropanol under reflux for 30 minutes. The resulting solution was cooled to 80° C., and the solution was filtered while hot. The filtered solution was then cooled to 5° C., and stirred for an additional 7 hours. The resulting crystalline precipitate was separated by filtering, followed by washing with methyl ethyl ketone. The recrystallized material was dried in vacuo at 45-85° C. Based upon HPLC-MS/MS analysis, the recrystallized tolperisone possessed 0.14% by weight 4-MMPPO.

### Example 2: Multiple Stage Recrystallization of Tolperisone

The approach utilized in Example 1 above was repeated with the exception that multiple stage (repeated) recrystallizations were carried out.

TABLE 1
4-MMPPO Content Following Repeated Recrystallizations,
Non-Acidified Solvent

| | | 4-MMPPO | 4-MMPPO | 4-MMPPO |
|---|---|---|---|---|
| | 4-MMPPO ppm | ppm After 1st | ppm After 2nd | ppm After 3rd |
| Batch | Crude Tolperisone | Recrystallization | Recrystallization | Recrystallization |
| 1 | 107/51.1 | <6.6 | 8.3 | <6.6 |
| | before/after | | | |
| | drying | | | |

As can be seen from the results in Table 1, multiple recrystallizations from solvents/solvent systems such as methyl ethyl ketone and isopropanol can be effective to provide tolperisone that is substantially absent 4-MMPPO.

### Example 3: Recrystallization of Tolperisone Followed by Acid Wash

Crude tolperisone was dissolved in an 85:15 mixture of 2-butanone (MEK) and isopropanol under reflux for 12 hours. The temperature was decreased to 80° C. and the solution filtered while hot. The filtered solution was cooled to 5° C. and stirred for 7 h at 5° C. The crystalline precipitate was separated by filtering and washed with a 1% hydrochloric acid/isopropanol mixture, and subsequently dried in vacuo at 45 to 85° C. Based upon HPLC-MS/MS analysis, a content of 4-MMPPO in the range of 1.5 to 10 ppm was detected in the recrystallized product.

### Example 4: Recrystallization of Tolperisone Using Acidified Solvent System

Crude tolperisone was dissolved in an 85:15 (v/v) mixture of 2-butanone (MEK) and isopropanol, accompanied by addition of 1% hydrochloric acid under reflux for 12 hours. The temperature was decreased to 80° C. and the solution filtered while hot. The solution was cooled to 5° C. and stirred for 7 h at 5° C. The crystalline precipitate was separated by filtering, washed with isopropanol, and subsequently dried in vacuo at 45 to 85° C., with lower drying temperatures being preferred. Tolperisone showed enhanced stability during recrystallization in the presence of acid. When 1% (v/v) concentrated aqueous hydrochloric acid was added to the recrystallization mixture, 4-MMPPO levels dropped below 6.6 ppm in the final recrystallized tolperisone product.

### Example 5: Multiple Recrystallizations of Tolperisone Using Acidified Solvent System

Crude tolperisone was dissolved in an 85:15 (v/v) mixture of 2-butanone (MEK) and isopropanol, accompanied by addition of 1% hydrochloric acid, under reflux for 12 hours. The temperature was decreased to 80° C. and the solution filtered while hot. The solution was cooled to 5° C. and stirred for 7 h at 5° C. The crystalline precipitate was separated by filtering, washed with isopropanol, and subsequently dried in vacuo at 45 to 85° C., with lower temperatures being preferred. The above recrystallization process was repeated 4 times.

TABLE 2
4-MMPPO Content Following Repeated Recrystallizations Using Acidified Solvent

| | | 4-MMPPO | 4-MMPPO | 4-MMPPO | 4-MMPPO |
|---|---|---|---|---|---|
| | 4-MMPPO ppm | ppm After 1st | ppm After 2nd | ppm After 3rd | ppm After 4th |
| Batch | Crude Tolperisone | Recrystallization | Recrystallization | Recrystallization | Recrystallization |
| 1 | 107/51.1 | <6.6 | <6.6 | <6.6 | <6.6 |
| | Before/after | | | | |
| | drying | | | | |

As can be seen from the results in Table 2, the first recrystallization under acidified conditions exhibited the most significant removal of 4-MMPPO and purification of tolperisone.

### Example 6: Instant Release Caplet Formulation

A solution of anhydrous citric acid, 2-butanone and isopropyl alcohol is prepared. Tolperisone hydrochloride containing less than 10 ppm 4-MMPPO as described herein is transferred into a granulator, into which the already prepared solution is placed. This mixture is homogenized and subsequently dried in a drier at 60° C., or more preferably, at 40° C. The formed granulate is sifted through a 1.8 mm screen. Silicon dioxide and talcum are added and likewise mixed. Subsequently, the mixture is further mixed with magnesium stearate. Tablets having a diameter of 8 mm and a weight of 155.8-172.2 g are produced. The finished granulate is coated with a suspension of hypromellose/hypromellose phthalate in ethanol/water, dyes and additives in a coating tank at a temperature of 55-60° C. The coated tablets are subsequently dried at room temperature.

### Example 7: Controlled Release Formulation

Tolperisone containing less than 10 ppm 4-MMPPO as described herein is granulated as the hydrochloride salt in a mixer with a solution consisting of Eudragit RS in butanone with addition of anhydrous citric acid. Subsequently, Eudragit S and Eudragit L are incorporated homogeneously; the mixture is dried and sifted. To the sifted granulate are added tabletting auxiliary agents, and the granulate is tableted. Tablets having a diameter of 8 mm and a weight of 190 mg are pressed. Subsequently, the tablets are coated (“filmed”) with a film material consisting of Eudragit L, dyes, and miscellaneous auxiliary agents, which are dissolved in butanol.

An example of a controlled release tablet is in Table 3a below, and an example of a coated immediate release tablet is in Table 3b below:

TABLE 3a

| Ingredient | Amount per tablet (mg) |
|---|---|
| Tolperisone hydrochloride | 150.00 |
| Eudragit RS | 1.88 |
| Eudragit L | 14.24 |
| Eudragit S | 10.50 |
| Aerosil | 1.80 |
| Stearic acid | 1.80 |
| Glycerol dibehenate | 7.50 |
| Iron oxide dye | 0.08 |
| Titanium dioxide | 4.08 |
| Talcum | 6.03 |
| Polyethylene glycol | 1.02 |
| Dimethylpolysiloxane | 0.05 |

TABLE 3b

| Ingredient | Amount per tablet (mg) | | | |
|---|---|---|---|---|
| Tolperisone hydrochloride | 50.00 | 100.00 | 150.00 | 200.00 |
| Citric acid, anhydrous | 10.00 | 20.00 | 30.00 | 40.00 |
| Lactose, anhydrous | 315.75 | 255.75 | 195.75 | 135.75 |
| Pregelatinized Starch | 22.50 | 22.50 | 22.50 | 22.50 |
| Crospovidone | 22.50 | 22.50 | 22.50 | 22.50 |
| Stearic acid | 6.75 | 6.75 | 6.75 | 6.75 |
| PEG 6000 | 22.50 | 22.50 | 22.50 | 22.50 |
| Opadry II 32F280008 | 13.50 | 13.50 | 13.50 | 13.50 |

### Example 8: Driving Simulation Study and Cognitive Function Test

Methodology: This was a randomized, placebo-controlled, multiple-dose 3-way cross-over study of the safety and cognitive effects of multiple doses of 150 mg tolperisone film-coated tablets (prepared according to methods described and referred to herein) administered three times daily (TID) in 31 male and female healthy volunteers. Treatment groups included 450 mg tolperisone (i.e., 150 mg tablets administered TID), 30 mg cyclobenzaprine (i.e., 10 mg tablets administered TID), and placebo. Cyclobenzaprine 10 mg tablets were procured by the individual study sites (Lot numbers 307574 and McKesson item number 554907/Manufacturing number 00378075101 were used for this study). Subjects received 3 days of each treatment over three treatment periods. Subject participation was approximately 3 weeks as outpatients with 3 days each week as overnight clinic participants. Subjects were screened within 28 days prior to Treatment Period 1 (Visit 2) for eligibility for participation in the study. Subjects who met all inclusion/exclusion criteria were admitted to the clinic on Day 1 to be evaluated for continued eligibility. Subjects performed a practice trial with the driving simulator and the cognitive function test. Subjects were dosed on the morning of Day 1. Approximately one hour after the second dose on Day 1, subjects were administered the cognitive test, followed by the driving simulator examination. On the morning of Day 2, prior to dosing, subjects were re-administered the cognitive test and driving examination to assess residual next day effects. Subjects repeated cognitive testing and the driving examination on the morning of Day 3, after administration of the AM study medication, to evaluate the cumulative effects of 3 days of dosing. Subjects were discharged on Day 3 with instructions to return to the clinic on Day 7 and Day 14 to repeat the above procedure with the second and third treatments. A follow-up phone call was conducted 1 week (±3 days) following discharge from the clinic on Day 17 to assess for continued safety. The total duration of study participation was approximately 4 weeks (range 4-8 weeks), including Screening and Follow-up.

Evaluation Criteria:

The pharmacodynamic measures included simulated driving performance as measured by SDLP using the Cognitive Research Corporation Driving Simulator-MiniSim (CRCDS-MiniSim), the Karolinska Sleepiness Scale, the CogScreen Symbol Digit Coding (SDC) test, a self-perceived safety to drive question, the Visual Analog Scale (VAS) to assess subject's motivation and self-appraisal of their driving performance, and an assessment of the relationship between single-dose plasma drug levels and driving performance. Blood samples for the determination of plasma tolperisone concentrations were drawn prior to each AM dosing on Days 1-3, 8-10, 15-17 and post-driving test (15-30 minutes after the drive) on Days 1, 8, and 15. Blood samples collected post-driving test on Days 3, 10, and 17 were drawn between 1.5 and 2 hours after the start of the drive (approximately 10:30 AM to 11:00 AM). Safety was assessed by the monitoring and recording of adverse events, vital sign measurements, clinical laboratory abnormalities, electrocardiograms (ECGs), pregnancy tests, and physical examinations.

Pharmacodynamic Analysis:

The primary endpoint, SDLP, was analyzed using a mixed-model, repeated measures with fixed effects for sequence, period, and treatment, with repeated observations for subjects. An unstructured covariance structure and Kenward-Roger degrees of freedom was used. In the event an unstructured covariance structure failed to converge, a variance components covariance structure was assumed. In addition, pair-wise within subject differences in SDLP greater than 4.4 cm in absolute value (equal to the previously found difference between placebo and 0.05% Blood Alcohol Content (BAC) for the CRCDS) were compared using McNemar's test. Pair-wise within subject differences in SDLP were also tested for symmetry about tolperisone using the maximally selected McNemar test. P-values for significance testing of period and sequence effects are also provided. Pair-wise comparisons (hypothesis tests) of differences in least squares means, and 95% confidence intervals on differences will be provided for:

Initial dose effects were measured at the following time-points:
cyclobenzaprine versus placebo following Day 1 PM dose;
tolperisone versus placebo following Day 1 PM dose and
tolperisone versus cyclobenzaprine following Day 1 PM dose.

Next day residual dose effects were measured at the following time-points:
cyclobenzaprine versus placebo preceeding Day 2 AM dose;
tolperisone versus placebo preceeding Day 2 AM dose; and
tolperisone versus cyclobenzaprine preceeding Day 2 AM dose.

Steady-state dose effects were measured at the following time-points:
cyclobenzaprine versus placebo following Day 3 AM dose;
tolperisone versus placebo following Day 3 AM dose; and
tolperisone versus cyclobenzaprine following Day 3 AM dose.

Summary statistics were provided (mean, SD, median, minimum, maximum) for SDLP for each time-point and treatment group. Data listings for driving simulator data were also provided.

Pharmacokinetic Analyses:

Blood samples for the determination of plasma tolperisone and cyclobenzaprine concentrations were drawn prior to each AM dosing on Days 1-3 and post-driving test (15-30 minutes after the drive) on Days 1 and 3. The relationship between initial dose effect, next day residual dose effect, and steady-state dose effect plasma drug levels and driving performance (i.e. the primary endpoint of SDLP) were assessed by correlation. Both the Spearman and Pearson correlations were reported.

Safety Data:

Safety analysis was based on the safety population. Safety measures were summarized using descriptive statistics and listed for each subject. MedDRA thesaurus was used to map adverse events verbatim to preferred terms and body systems. WHOdrug thesaurus (Sep. 1, 2016) was used to map prior medication and concomitant medication verbatim to preferred terms and ATC Class.

### Test Details:

### CRCDS Country Vigilance-Divided Attention (CVDA) Driving Scenario on the CRCDS-MiniSim

A 62.1 mile (100 km), monotonous, two-lane highway driving task that includes a secondary visual vigilance task (DA). The monotonous Country Vigilance scenario has been demonstrated to be sensitive to detect the effects of fatigue or sleepiness on driving performance. This scenario has been useful in measuring the effects of sleep deprivation, Obstructive Sleep Apnea, chronic primary insomnia, and is sensitive to CNS depressants (e.g., alcohol and sedating antihistamines). Results obtained using this methodology are comparable to those obtained using over-the-road driving tests. Driving performance endpoints include:
Lane exceedance; including number, maximum, and duration;
Average speed, speed deviation, speedings count, speedings ratio;
Excessive Ay (cornering speed threshold exceeded);
Total collisions (i.e., lane exceedances >4 feet); and
Divided Attention Measures: Correct Responses, Omission Errors, Commission Errors, Reaction Time, Standard Deviation of Reaction Times.

### Digit Symbol Substitution Test (CogScreen Symbol Digit Coding)

This test is used to measure attention, visual scanning, working memory, and speed of information processing. SDC is a computer analogue of the conventional symbol-substitution task found in the WAIS-R Digit Symbol subtest and the Symbol Digit Modalities Test. Details of the use of the SDC are provided in the CogScreen® Examiner Manual, CogScreen LLC, 2016. CogScreen SDC endpoints include:
Number of correct responses;
Response Accuracy; and
Standard deviation of reaction time.

### Karolinska Sleepiness Scale (KSS) Test

The Karolinska Sleepiness Scale (KSS) was used to assess subjective level of sleepiness. This is a subject self-report measure of situational sleepiness and provides an assessment of alertness/sleepiness at a particular point in time. The KSS has been found to correlate with electroencephalogram and behavioral variables. Subjects indicated their level of alertness versus sleepiness according to the following scale:

(1) “extremely alert”

(2)

(3) “alert”

(4)

(5) “neither sleepy nor alert”

(6)

(7) “sleepy”

(8)

(9) “extremely sleepy-fighting sleep”

The KSS assessment was performed immediately before and after the simulated driving test Subjects self-reported their KSS assessments and the scores were recorded in the case report form.

### Safety Measures

The Principal Investigator was responsible for the monitoring of subjects' safety and reporting of all adverse events (AEs) and serious adverse events (SAEs). The study personnel reviewed subject-volunteered information related to subjective complaints, and reviewed all laboratory findings and queried the subject specifically for visits requiring hospitalization, procedures, laboratory follow-up, or unscheduled visits to see their physician. Each trial subject was questioned about AEs during treatment with study drug. All AEs were documented in the subject's source documents as well as on the appropriate case report form (CRF). The Investigator assessed and recorded any AE in detail on the AE CRF pages including a description of the event, the date of onset, severity, time course, duration and outcome, relationship of the AE to study drug, and an alternate etiology for events not considered “probably” related to study drug and any actions taken.

### Results:

In the following tables, a p value >0.05 indicates no difference in effect of drug versus placebo that is statistically significant.

FIG. 1 is a bar chart comparing the SDLP data for tolperisone, cyclobenzaprine and placebo. The data used to generate the bar chart is provided in Table 4 below:

TABLE 4
Standard Deviation of Lateral Position (SDLP)

| | | Tolperisone | Cyclobenzaprine | Tolperisone | Cyclobenzaprine | Cyclobenzaprine |
|---|---|---|---|---|---|---|
| | Placebo | 450 mg | 30 mg | vs. Placebo | vs. Placebo | vs. Tolperisone |
| Day 1 | 29.6 | 29.5 | 38.1 | p = 0.9 | p = <0.0001 | p = <0.0001 |
| (at Tmax) | | | | | | |
| Next Day | 29.8 | 29.8 | 35.0 | p = 0.71 | p = <0.0001 | p = <0.0001 |
| Residual | | | | | | |
| Steady | 29.5 | 29.7 | 32.0 | p = 0.83 | p = <0.0015 | p = <0.0027 |
| State | | | | | | |

FIG. 2 compares the tolperisone and cyclobenzaprine SDLP mean differences from placebo.

FIG. 3 compares the tolperisone and cyclobenzaprine mean blood plasma concentrations over time. Correlational analyses generally showed no relationship between plasma concentrations of tolperisone and measures of driving performance, cognitive functioning, or on self-report measures. The one exception for tolperisone was a significant relationship between performance on the Symbol Digit Coding Test and plasma concentration, such that higher concentrations were associated with better performance on the test. For cyclobenzaprine, significant relationships were found on Day 2 between plasma level and Total Collisions and between plasma level and self-reported sleepiness, both indicating significant residual next-day effects.

Table 5 below presents comparative data on the Digit Symbol Substitution Test (CogScreen Symbol Digit Coding).

TABLE 5
Symbol Digit Coding (SDC) Cognitive Research Version

| | | Tolperisone | Cyclobenzaprine | Tolperisone | Cyclobenzaprine | Cyclobenzaprine |
|---|---|---|---|---|---|---|
| | Placebo | 450 mg | 30 mg | vs. Placebo | vs. Placebo | vs. Tolperisone |
| Day 1 | 67.0 | 66.6 | 66.4 | p = 0.86 | p = 0.81 | p = 0.94 |
| (at Tmax) | | | | | | |
| Next Day | 68.8 | 69.3 | 66.7 | p = 0.35 | p = 0.07 | p = 0.09 |
| Residual | | | | | | |
| Steady | 68.9 | 70.1 | 67.4 | p = 0.29 | p = 0.36 | p = 0.05 |
| State | | | | | | |

Tables 6 and 7 below are data outputs from the CVDA driving scenario on the CRCDS-MiniSim.

TABLE 6
Lane Exceedance (extent of driving outside of lanes)

| | | Tolperisone | Cyclobenzaprine | Tolperisone | Cyclobenzaprine | Cyclobenzaprine |
|---|---|---|---|---|---|---|
| | Placebo | 450 mg | 30 mg | vs. Placebo | vs. Placebo | vs. Tolperisone |
| Day 1 | 2.4 | 2.4 | 3.6 | p = 0.99 | p = <0.0001 | p = <0.0001 |
| (at Tmax) | | | | | | |
| Next Day | 2.2 | 2.1 | 3.2 | p = 0.90 | p = <0.0001 | p = <0.0001 |
| Residual | | | | | | |
| Steady | 2.4 | 2.3 | 2.9 | p = 0.43 | p = 0.0167 | p = 0.0019 |
| State | | | | | | |

TABLE 7
Total Collisions

| | | Tolperisone | Cyclobenzaprine | Tolperisone | Cyclobenzaprine | Cyclobenzaprine |
|---|---|---|---|---|---|---|
| | Placebo | 450 mg | 30 mg | vs. Placebo | vs. Placebo | vs. Tolperisone |
| Day 1 | 0.2 | 0.0 | 1.6 | p = 0.50 | p = 0.0938 | p = 0.0625 |
| (at Tmax) | | | | | | |
| Next Day | 0.1 | 0.0 | 0.4 | p = 1.00 | p = 0.250 | p = 0.125 |
| Residual | | | | | | |
| Steady | 0.1 | 0.0 | 0.1 | p = 0.50 | p = 1.00 | p = 1.00 |
| State | | | | | | |

Additional secondary driving endpoints were also measured. Table 8 shows the p-values for treatment comparisons for the secondary driving endpoints.

TABLE 8
Secondary Driving Endpoints: P-
Values for Treatment Comparisons

| | Tol v Placebo | Tol v Cyc | Cyc v Placebo |
|---|---|---|---|
| Maintenance of | | | |
| Lane Position | | | |
| Lane Exceedance | 0.8181 | <0.0001 | <0.0001 |
| Number | | | |
| Lane Exceedance | 0.4127 | 0.0009 | 0.0119 |
| Maximum | | | |
| Lane Exceedance | 0.8064 | <0.0001 | <0.0001 |
| Duration | | | |
| Speed Control | | | |
| Average Speed | 0.6751 | 0.6716 | 0.3871 |
| Speed Deviation | 0.1533 | <0.0001 | 0.0009 |
| Speedings Count | 0.6587 | 0.0668 | 0.0207 |
| Speedings Ratio | 0.8478 | 0.0500 | 0.0272 |
| Divided Attention | | | |
| Correct Responses | 0.2135 | 0.8740 | 0.1537 |
| Omission Errors | 0.2719 | 0.6551 | 0.1196 |
| Commission Errors | 0.9297 | 0.1659 | 0.1903 |
| Reaction Time (RT) | 0.0155 | 0.0001 | 0.1130 |
| Std Deviation of RT | 0.0342 | 0.0693 | 0.7215 |
| Other Safety | | | |
| Related Measures | | | |
| Total collisions | 0.5000 | 0.0625 | 0.0938 |
| Excessive Ay | 0.7683 | 0.0309 | 0.0148 |

### Lane Exceedance

The principal measure of lane exceedance is the number of lane exceedances, an indication of lane position control, (i.e., the driver's ability to stay within their lane), as measured by the number of times that the front left or right tire of the vehicle crosses over the right or left lane boundary. There was a significantly higher number of lane exceedances following cyclobenzaprine (Cyc) treatment compared to placebo (p<0.001 on Days 1 and 2, p=0024 on Day 3). There was no significant difference in the number of lane exceedances between tolperisone (Tol) and placebo (p=0.818, p=0.849, p=0.524; Days 1, 2, and 3 respectively). The difference between Tol and Cyc with respect to number of lane exceedances was significant at all three time-points (p<0.001 for Days 1 and 2; p=0.024, Day 3). The severity of lane exceedance is assessed by related measures, including Lane Exceedance Maximum (i.e., the maximum lateral deviation that the vehicle traveled from the lane center) and Duration of Exceedance (i.e., the amount of time that it took for the driver to make corrections to bring the vehicle back into the lane of travel). For these two measures, performance was significantly worse for Cyc compared to placebo on Days 1 and 2. By contrast, significant increases in Lane Exceedance Maximum and Duration of Exceedance were not seen for Tol compared to placebo at any time-point.

### Speed Related Measures (Average Speed, Speed Deviation, Excessive Speed Count and Speedings Ratio)

Speed Deviation, the speed measure which is sensitive to sedating drug effects, showed significantly higher speed variability for Days 1 and 2 (p<0.001 and p=0.0015; respectively) for Cyc compared to placebo. The impact of Tol on speed variability was non-significant on all three test days (p>0.153) when compared to placebo. The additional speed control measures primarily assess the tendency for high speed driving behavior and are typically less sensitive to sedation. Average speed did not differ significantly from placebo for Tol or for Cyc. On the other hand, Speedings Count (i.e., the number of times that the driver exceeded the posted 55 mph speed limit by 7 mph) was not significantly impacted by Tol but was significant for Cyc compared to placebo (p=0.021, p=0.029, p=0.033; Day 1, 2, and 3, respectively). An additional measure of compliance with the speed limit, Speedings Ratio, is obtained by calculating the percent of time driven more than 5.5 mph over the speed limit relative to the total drive time. Tol had no significant effect on Speedings Ratio compared to placebo. In contrast, there was a significant effect of Cyc on Speedings Ratio limited to Day 1 (p=0.027). For Day 2 and 3 there was no significant effect of Cyc on Speedings Ratio.

### Other Driving Safety Measures: Excessive Ay and Collisions

Excessive speed in corners (Excessive Ay), as measured by lateral g-force (i.e., exceeding 0.25 g), is included in driving studies as a measure of general driving safety. Following dosing with Tol there was no significant increase in Excessive Ay compared to placebo. In contrast, there was a significant effect of Cyc on Excessive Ay on Day 1 (p=0.015) and a trend on Day 2 (p=0.098). Total Collisions, defined as the vehicle wholly drifting outside the lane, represents the sum of collisions with other vehicles and off-road crashes as well as the number of times that lane deviation exceeded 4 feet (i.e., a crash-likely event). There was no significant increase in Total Collisions with Tol or Cyc compared to placebo, though a trend for increased Total Collisions for Cyc compared to placebo was seen on Day 1 (p=0.094).

### Divided Attention (Correct Responses, Omission Errors, Commission Errors, Reaction Time, and Standard Deviation of Reaction Time):

The CRCDS driving scenarios include an embedded measure of multitasking or divided attention. The divided attention task generates measures of accuracy (i.e., correct responses, omission errors, commission errors, percent accuracy) and measures of response speed (i.e., reaction time and reaction time variability). For Tol and Cyc there was no significant effect of treatment on the number of Correct Responses compared to placebo. Analysis of the number of Omission Errors (i.e., lapses of attention) showed no increase in these errors following treatment with Tol or Cyc. Analysis of the number of Commission Errors (i.e., failures of response inhibition) also shows non-significant treatment group differences for Tol and Cyc compared to placebo.

Reaction time on the divided attention test is a measure of the time elapsed from the appearance of the divided attention target stimulus until the subject's response. For this measure of reaction time, subjects responded significantly faster on Day 1 when treated with Tol compared to placebo (p=0.0155). None of the other treatment comparisons for Tol or Cyc are significant, however the difference between Tol and Cyc is significant for Day 1 (p<0.001) and approached significance on Day 2 (p=0.099). Reaction time variability (i.e., Standard Deviation of Reaction Time) showed essentially the same treatment effects, with significantly decreased variability following Tol on Day 1 (p=0.034) but no other treatment group differences.

### Karolinska Sleepiness Scale (KSS) Test

In the KSS test subjects reported significantly more subjective sleepiness following dosing with cyclobenzaprine on Day 1 (p<0.001). Subjects did not report feeling more sleepy with cyclobenzaprine on Day 2 or Day 3. Following dosing with tolperisone, there was no significant increase in self-reported sleepiness at any time-point. Significantly more sleepiness was reported following cyclobenzaprine than following tolperisone on Day 1 (p<0.001) and the same trend was observed on Day 3. The detailed KSS results are presented in Table 9 below.

TABLE 9
Karolinska Sleepiness Scale (KSS)

| | | Tolperisone | Cyclobenzaprine | Tolperisone | Cyclobenzaprine | Cyclobenzaprine |
|---|---|---|---|---|---|---|
| | Placebo | 450 mg | 30 mg | vs. Placebo | vs. Placebo | vs. Tolperisone |
| Day 1 | 3.4 | 3.5 | 5.5 | p = 0.57 | p = <0.0001 | p = <0.0001 |
| (at Tmax) | | | | | | |
| Next Day | 3.9 | 4.0 | 4.1 | p = 0.66 | p = 0.83 | p = 0.82 |
| Residual | | | | | | |
| Steady | 3.3 | 3.1 | 3.8 | p = 0.71 | p = 0.19 | p = 0.100 |
| State | | | | | | |

### Safety

A total of 49 Treatment Emergent Adverse Events (TEAEs) were reported by 21 subjects during the study. The treatment with the highest number of TEAEs reported was cyclobenzaprine (n=21), followed by tolperisone (n=15) and placebo (n=13). The majority of the TEAEs were mild or moderate in intensity. There was one severe AE of dizziness reported by a subject during the cyclobenzaprine treatment arm. Overall, 4 subjects discontinued before completing the study, including one subject due to a moderate AE of a Crohn's Disease flare up following the first day of dosing in the tolperisone treatment arm and 3 subjects who withdrew consent. There were no SAEs reported during this study. Table 10 below lists the recorded treatment-related adverse events.

TABLE 10
Treatment Emergent Adverse Events

| | | Tolperisone | Cyclobenzaprine |
|---|---|---|---|
| | Placebo | 450 mg | 30 mg |
| Cardiac | 0 | 0 | 1 |
| Gastrointestinal | 3 | 1 | 4 |
| Disorders | | | |
| General: | 0 | 0 | 1 |
| Sluggishness | | | |
| Nervous System | | | |
| Disorders | | | |
| Attention | 0 | 1 | 0 |
| Dizziness | 2 | 1 | 3 |
| Headache | 0 | 1 | 0 |
| Lethargy | 0 | 0 | 1 |
| Somnolence | 2 | 5 | 10 |
| Psychiatric | 0 | 0 | 1 |
| (Insomnia) | | | |
| Respiratory | 0 | 1 | 0 |
| Skin | 1 | 0 | 0 |

1. A method of treating a patient with muscle spasm or spastic syndrome, wherein based on the patient's routine daily activities, an optimal daily treatment regimen of tolperisone or a pharmaceutically acceptable salt thereof is selected to treat muscle spasm or spastic syndrome without impairing cognitive function.

2. The method of claim 1, wherein one of the patient's routine daily activities is driving.

3. The method of claim 1, wherein the patient's cognitive function is determined by a simulated driving test.

4. The method of claim 3, wherein the patient's Standard Deviation of Lateral Position (SDLP) score from the simulated driving test after tolperisone administration is substantially the same as the patient's SDLP score without tolperisone administration.

5. The method of claim 3, wherein the patient's lane exceedance score from the simulated driving test after tolperisone administration is substantially the same as the patient's lane exceedance score without tolperisone administration.

6. The method of claim 1, wherein cognitive function is determined by the patient's score on the Karolinska Sleepiness Scale.

7. The method of claim 1, wherein the treatment regimen of tolperisone or a pharmaceutically acceptable salt thereof is selected to treat muscle spasm or spastic syndrome and provides a patient Karolinska Sleepiness Scale score of about 4 or less.

8. A method of treating a patient with muscle spasm or spastic syndrome comprising (1) assessing the patient's routine daily activities, (2) determining if one or more of said activities could be, or is/are being, compromised by administering a drug effective to treat muscle spasm or spastic syndrome, but also known to have a sedative effect and (3) substituting said drug with tolperisone or a pharmaceutically acceptable salt thereof, and administering tolperisone or a pharmaceutically acceptable salt thereof in an amount effective to treat muscle spasm or spastic syndrome without compromising the patient's routine daily activities.

9. The method of claim 8, wherein one of the patient's routine daily activities is driving.

10. The method of claim 8, wherein the amount of tolperisone or pharmaceutically acceptable salt thereof administered is effective to treat muscle spasm or spastic syndrome without impairing the patient's cognitive function.

11. The method of claim 10, wherein the patient's cognitive function is determined by a simulated driving test.

12. The method of claim 11, wherein the patient's Standard Deviation of Lateral Position (SDLP) score from the simulated driving test after tolperisone administration is substantially the same as the patient's SDLP score without tolperisone administration.

13. The method of claim 11, wherein the patient's lane exceedance score from the simulated driving test after tolperisone administration is substantially the same as the patient's lane exceedance score without tolperisone administration.

14. The method of claim 10, wherein cognitive function is determined by the patient's score on the Karolinska Sleepiness Scale.

15. The method of claim 8, wherein the amount of tolperisone or pharmaceutically acceptable salt thereof administered is effective to treat muscle spasm or spastic syndrome and provides a patient Karolinska Sleepiness Scale score of about 4 or less.

16. A method of treating a patient with muscle spasm or spastic syndrome, comprising administering to the patient a therapeutically effective amount of tolperisone or a pharmaceutically acceptable salt thereof, and wherein the patient was previously treated with a muscle relaxant but treatment was discontinued because of the muscle relaxant's sedative effect.

17. The method of claim 16, wherein the muscle relaxant is cyclobenzaprine hydrochloride.

18. A method of treating a patient with muscle spasm or spastic syndrome, comprising administering to the patient a therapeutically effective amount of tolperisone or a pharmaceutically acceptable salt thereof, and wherein an improvement is also seen in the patient's cognitive function.

19. The method of claim 18, wherein the patient demonstrates an improvement in alertness.

20. The method of claim 18, wherein the patient demonstrates an improvement in time to react to a situation.

21. The method of claim 18, wherein the patient demonstrates an improvement in time to react during driving.

22. The method of claim 1, wherein tolperisone or pharmaceutically acceptable salt thereof is administered three times daily to a total daily dose of about 150 mg to about 900 mg.

23. The method of claim 22, wherein tolperisone is administered in 50 mg, 100 mg, 150 mg or 200 mg unit doses to a total daily dose of 150 mg to 600 mg.

24. The method of claim 1, wherein tolperisone is administered as racemic tolperisone hydrochloride.

25. The method of claim 1, wherein the patient is administered racemic tolperisone hydrochloride and receives a total daily exposure of less than 1.5 μg 2-methyl-1-(4-methylphenyl)-propenone (4-MMPPO).

26. The method of claim 1, wherein tolperisone or pharmaceutically acceptable salt thereof is administered to treat a patient with painful muscle spasm of the back or neck.

27. The method of claim 26, wherein the patient is also administered a pain medication.

28. The method of claim 27, wherein the pain medication is a mild analgesic drug.

29. The method of claim 27, wherein the pain medication is an opioid drug, where the opioid drug is administered at a reduced dose compared to the dose required to effectively treat a patient with painful muscle spasm of the back or neck who is not also administered tolperisone or pharmaceutically acceptable salt thereof.

30. A method of determining the suitability of a drug for treating a patient suffering from muscle spasm or spastic syndrome, comprising conducting a driving simulation study and comparing the sedative effect of the drug against a standard therapy for treating muscle spasm or the spastic syndrome.

31. The method of claim 30, wherein the standard therapy for treating muscle spasm or the spastic syndrome is treatment with cyclobenzaprine hydrochloride.
